(19) United States
(12) Patent Application Publication
(10) Pub. No.: US 2024/0293732 A1
(43) Pub. Date: Sep. 5, 2024

(54) SYSTEM, METHOD AND COMPUTER PROGRAM FOR MONITORING A WEIGHT DISTRIBUTION OF A USER

(71) Applicant: Sony Interactive Entertainment Inc., Tokyo (JP)

(72) Inventors: Paul Terence Mulligan, London (GB); Jun Leung, London (GB)

(73) Assignee: Sony Interactive Entertainment Inc., Tokyo (JP)

(21) Appl. No.: 18/585,143

(22) Filed: Feb. 23, 2024

(30) Foreign Application Priority Data
Mar. 2, 2023 (GB) 2303078.6

(51) Int. Cl. A63F 13/218; A63F 13/285; A63F 13/533

(52) U.S. Cl. CPC A63F 13/218; A63F 13/285; A63F 13/533

(57) ABSTRACT

A system for monitoring a weight distribution of a user is provided, the system includes: one or more sensors configured to sense a weight distribution of a user; circuitry configured to analyse a change of weight distribution of the user to determine a posture of the user; and one or more feedback devices configured to provide feedback related to the posture of the user when the change in weight distribution of the user satisfies a predetermined condition.

### BACKGROUND OF THE INVENTION

### Field of the invention

The present disclosure relates to a system, method and computer program for monitoring a weight distribution of a user.

### Description of the Prior Art

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present invention.

In recent years, the use of devices, such as an information processing apparatuses, has become more widespread. In particular, a significant number of tasks now require the use of an information processing apparatus. This can include tasks in the workplace, for example. An information processing apparatus is now also often used also in a home environment. For example, a person may use an information processing apparatus for entertainment purposes such as playing video games or otherwise interacting with digital content (including editing a video, viewing information on the internet or the like).

Therefore, a user may often spend a significant portion of their time using an information processing apparatus. During this time, a user may assume a certain posture or position and/or may remain relatively sedentary. This can become uncomfortable for the user.

A user may also assume a certain position and/or remain relatively sedentary when performing other tasks or operations (such as when driving a vehicle).

Accordingly, there is a risk that a user may experience a certain level of discomfort when using an information processing apparatus or performing other tasks or operations.

It is an aim of the present disclosure to address or mitigate this problem.

### SUMMARY OF THE INVENTION

A brief summary about the present disclosure is provided hereinafter to provide basic understanding related to certain aspects of the present disclosure.

Embodiments of the present disclosure are defined by the independent claims. Further aspects of the disclosure are defined by the dependent claims.

In accordance with embodiments of the disclosure, the weight distribution of a user using a device can be monitored. Accordingly, it is possible to verify that a user is correctly positioned when using a device. Moreover, by providing feedback to the user, it becomes easier for a user to correct their posture or position when using the device. Thus, comfort of the user when using an information processing apparatus, using a device and/or performing other tasks and operations can be improved.

Of course, it will be appreciated that the present disclosure is not particularly limited to these advantageous technical effects. Other advantageous technical effects will become apparent to the skilled person when reading the disclosure.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates an apparatus in accordance with embodiments of the disclosure;

FIG. 2 illustrates an example of an entertainment system in accordance with embodiments of the disclosure;

FIG. 3 illustrates an example handheld controller in accordance with embodiments of the disclosure;

FIG. 4 illustrates the architecture of an example HMD device in accordance with embodiments of the disclosure;

FIG. 5 illustrates an example system in accordance with embodiments of the disclosure;

FIG. 6 illustrates an example sensor configuration in accordance with embodiments of the disclosure;

FIGS. 7A and 7B illustrate an example weight distribution in accordance with embodiments of the disclosure;

FIGS. 7C illustrates an example change in weight distribution in accordance with embodiments of the disclosure;

FIG. 8 illustrates an example method in accordance with embodiments of the disclosure.

### DESCRIPTION OF THE EMBODIMENTS

The foregoing paragraphs have been provided by way of general introduction, and are not intended to limit the scope of the following claims. The described embodiments, together with further advantages, will be best understood by reference to the following detailed description taken in conjunction with the accompanying drawings (wherein like reference numerals designate identical or corresponding parts throughout the several views).

In the following description, a number of specific details are presented in order to provide a thorough understanding of embodiments of the present disclosure. It will be apparent, however, to a person skilled in the art that these specific details need not be employed to practice the invention. Conversely, specific details known to the person skilled in the art are omitted for the purposes of clarity where appropriate.

Furthermore, the terms “coupled” and “connected,” along with their derivatives, may be used herein to describe structural relationships between components of the apparatus or system for performing the operations herein. It should be understood that these terms are not intended as synonyms for each other. Rather, in particular embodiments, “connected” is used to indicate that two or more elements are in direct physical or electrical contact with each other while “coupled” is used to indicate two or more elements are in either direct or indirect (with other intervening elements between them) physical or electrical contact with each other, and/or that the two or more elements co-operate or communicate with each other (e.g., as in a cause and effect relationship).

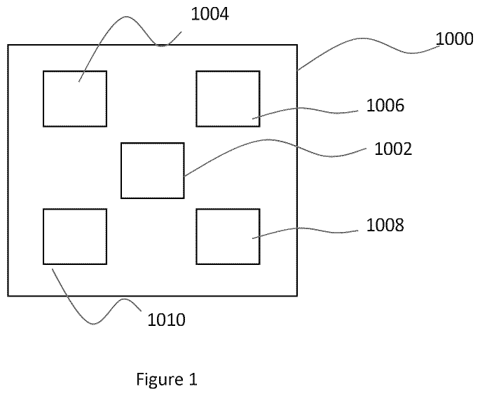

Referring to FIG. 1, an apparatus 1000 (an example of an information processing apparatus) according to embodiments of the disclosure is shown. Typically, an apparatus 1000 according to embodiments of the disclosure is a computer device such as a personal computer, an entertainment system or videogame console such as the Sony® PlayStation 5®, or a terminal connected to a server. Indeed, in embodiments, the apparatus may also be a server. The apparatus 1000 is controlled using a microprocessor or other processing circuitry 1002. In some examples, the apparatus 1000 may be a portable computing device such as a mobile phone, laptop computer or tablet-computing device.

The processing circuitry 1002 may be a microprocessor carrying out computer instructions or may be an Application Specific Integrated Circuit. The computer instructions are stored on storage medium 1004 which maybe a magnetically readable medium, optically readable medium or solid state type circuitry. The storage medium 1004 may be integrated into the apparatus 1000 or may be separate to the apparatus 1000 and connected thereto using either a wired or wireless connection. The computer instructions may be embodied as computer software that contains computer readable code which, when loaded onto the processor circuitry 1002, configures the processor circuitry 1002 to perform a method according to embodiments of the disclosure.

Additionally, an optional user input device 1006 is shown connected to the processing circuitry 1002. The user input device 1006 may be a touch screen or may be a mouse or stylist type input device. The user input device 1006 may also be a keyboard, controller, or any combination of these devices.

A network connection 1008 may optionally be coupled to the processor circuitry 1002. The network connection 1008 may be a connection to a Local Area Network or a Wide Area Network such as the Internet or a Virtual Private Network or the like. The network connection 1008 may be connected to a server allowing the processor circuitry 1002 to communicate with another apparatus in order to obtain or provide relevant data. The network connection 1002 may be behind a firewall or some other form of network security.

Additionally, shown coupled to the processing circuitry 1002, is a display device 1010. The display device 1010, although shown integrated into the apparatus 1000, may additionally be separate to the apparatus 1000 and may be a monitor or some kind of device allowing the user to visualize the operation of the system (e.g. a display screen or a head mounted display). In addition, the display device 1010 may be a printer, projector or some other device allowing relevant information generated by the apparatus 1000 to be viewed by the user or by a third party.

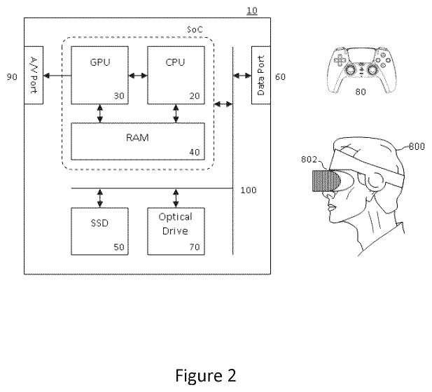

Referring now to FIG. 2, an example of an entertainment system in accordance with embodiments of the disclosure is illustrated. An example of an entertainment system 10 is a computer or console such as the Sony® PlayStation 5® (PS5). The entertainment system 10 is an example of an information processing apparatus 1000 in accordance with embodiments of the disclosure.

The entertainment system 10 comprises a central processor 20. This may be a single or multi core processor, for example comprising eight cores as in the PS5. The entertainment system also comprises a graphical processing unit or GPU 30. The GPU can be physically separate to the CPU, or integrated with the CPU as a system on a chip (SoC) as in the PS5.

The entertainment device also comprises RAM 40, and may either have separate RAM for each of the CPU and GPU, or shared RAM as in the PS5. The or each RAM can be physically separate, or integrated as part of an SoC as in the PS5. Further storage is provided by a disk 50, either as an external or internal hard drive, or as an external solid state drive, or an internal solid state drive as in the PS5.

The entertainment device may transmit or receive data via one or more data ports 60, such as a USB port, Ethernet® port, WiFi® port, Bluetooth® port or similar, as appropriate. It may also optionally receive data via an optical drive 70.

Interaction with the system is typically provided using one or more handheld controllers 80, such as the DualSense® controller in the case of the PS5.

Audio/visual outputs from the entertainment device are typically provided through one or more A/V ports 90, or through one or more of the wired or wireless data ports 60.

Where components are not integrated, they may be connected as appropriate either by a dedicated data link or via a bus 100.

An example of a device for displaying images output by the entertainment system is a head mounted display ‘HMD’ 802, worn by a user 800.

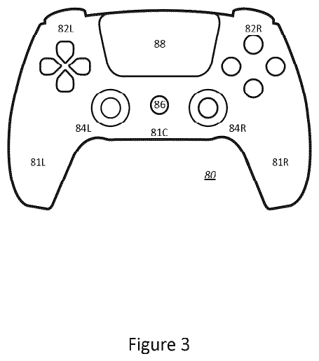

Turning now to FIG. 3 of the present disclosure, an example of a handheld controller in accordance with embodiments of the disclosure is illustrated. Indeed, in FIG. 3, a DualSense® controller 80 is illustrated as an example of a handheld controller. Such a controller typically has two handle sections 81L,R and a central body 81C. Various controls are distributed over the controller, typically in local groups. Examples include a left button group 82L, which may comprise directional controls and/or one or more shoulder buttons, and similarly right button group 82R, which comprise function controls and/or one or more shoulder buttons. The controller also includes left and/or right joysticks 84L,R, which may optionally also be operable as buttons by pressing down on them.

The controller (typically in the central portion of the device) may also comprise one or more system buttons 86, which typically cause interaction with an operating system of the entertainment device rather than with a game or other application currently running on it; such buttons may summon a system menu, or allow for recording or sharing of displayed content. Furthermore, the controller may comprise one or more other elements such as a touchpad 88, a light for optical tracking (not shown), a screen (not shown), haptic feedback elements (not shown), and the like.

It will be appreciated that a head mounted display ‘HMD’, worn by a user, can display images output by the entertainment system.

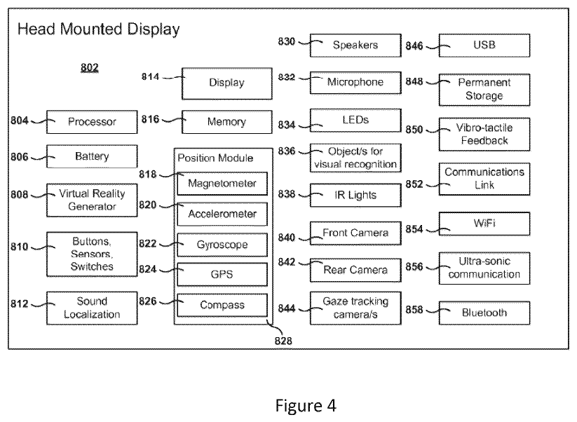

Referring now to FIG. 4, this illustrates the architecture of an example HMD device. The HMD may also be a computing device and may include modules usually found on a computing device, such as one or more of a processor 804, memory 816 (RAM, ROM, etc.), one or more batteries 806 or other power sources, and permanent storage 848 (such as a solid state disk).

One or more communication modules can allow the HMD to exchange information with other portable devices, other computers (e.g. the PS5®), other HMDs, servers, etc. Communication modules can include a Universal Serial Bus (USB) connector 846, a communications link 852 (such as Ethernet®), ultrasonic or infrared communication 856, Bluetooth® 858, and WiFi® 854.

A user interface can include one or more modules for input and output. The input modules can include input buttons (e.g. a power button), sensors and switches 810, a microphone 832, a touch sensitive screen (not shown, that may be used to configure or initialize the HMD), one or more front cameras 840, one or more rear cameras 842, one or more gaze tracking cameras 844. Other input/output devices, such as a keyboard or a mouse, can also be connected to the portable device via communications link, such as USB or Bluetooth®.

The output modules can include the display 814 for rendering images in front of the user's eyes. Some embodiments may include one display, two displays (one for each eye), micro projectors, or other display technologies. Other output modules can include Light-Emitting Diodes (LED) 834 (which may also be used for visual tracking of the HMD), vibro-tactile feedback 850, speakers 830, and a sound localization module 812, which performs sound localization for sounds to be delivered to speakers or headphones. Other output devices, such as headphones, can also connect to the HMD via the communication modules, be permanently attached to the HMD, or integral to it.

One or more elements that may be included to facilitate motion tracking include LEDs 834, one or more objects for visual recognition 836, and infrared lights 838. Alternatively or in addition, the one or more front or rear cameras may facilitate motion tracking based on image motion.

Information from one or more different modules can be used by the position module 828 to calculate the position of the HMD. These modules can include a magnetometer 818, an accelerometer 820, a gyroscope 822, a Global Positioning System (GPS) module 824, and a compass 826. Alternatively or in addition, the position module can analyze image data captured with one or more of the cameras to calculate the position. Further yet, optionally the position module can perform tests to determine the position of the portable device or the position of other devices in the vicinity, such as a WiFi ping test or ultrasound tests.

A virtual reality generator 808 then outputs one or more images corresponding to a virtual or augmented reality environment or elements thereof, using the position calculated by the position module. The virtual reality generator 808 may cooperate with other computing devices (e.g., PS5® or other game console, Internet server, etc.) to generate images for the display module 814. The remote devices may send screen updates or instructions for creating game objects on the screen. Hence the virtual reality generator 808 may be responsible for none, some, or all of the generation of one or more images then presented to the user, and/or may be responsible for any shifting of some or all of one or both images in response to inter-frame motion of the user (e.g. so-called reprojection).

It should be appreciated that the embodiment illustrated in FIG. 4 is an exemplary implementation of an HMD, and other embodiments may utilize different modules, a subset of the modules, or assign related tasks to different modules. The embodiment illustrated in FIG. 4 should therefore not be interpreted to be exclusive or limiting, but rather exemplary or illustrative. It will also be appreciated that the functionality of at least a subset of the modules may be provided by, or in concert with, corresponding modules of the entertainment device (in turn typically provided by a processor of that device operating under suitable software instruction).

Consider, now, a situation where a user is using an information processing apparatus, such as the entertainment system 10 as described with reference to FIG. 2 of the present disclosure. During use of the entertainment system, the user may assume a certain posture or position. For example, the user may sit on a chair while using the entertainment system. Alternatively, the user may stand in a certain position when using the entertainment system. If the user does not assume a correct posture or position for using the entertainment system, then the user may become uncomfortable. It may therefore become difficult for the user to continue using the entertainment system.

Furthermore, if the user is using a HMD while using the entertainment system, certain content displayed on the HMD may make it more difficult for the user to balance (e.g. if the orientation of the image shown on the HMD is not aligned with the ground on which the user is standing). This can make the user uncomfortable.

The user may also become uncomfortable if they do not assume a correct posture or position when using a different object or device. For example, a user travelling in a vehicle (such as an electric car) may become uncomfortable if they do not assume a correct posture or position when travelling in the vehicle. It may therefore become difficult for the user to continue travelling in the vehicle. Alternatively, for example, a user may become uncomfortable if they do not assume a correct posture or position when using an exercise machine or other fitness equipment. Accordingly, it may be difficult for a user to continue using the exercise machine or other fitness equipment.

Accordingly for at least these reasons, in addition to those reasons described in the Background, a system, method and computer program are provided.

### System

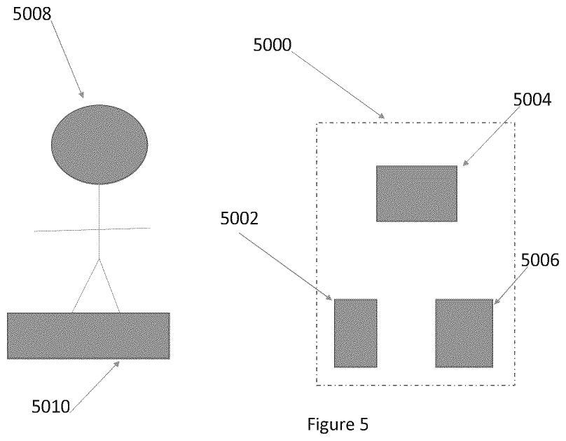

Turning to FIG. 5 of the present disclosure, the configuration of a system according to embodiments of the disclosure is illustrated.

In particular, FIG. 5 of the present disclosure illustrates an example of a system 5000 for monitoring the weight distribution of a user in accordance with embodiments of the disclosure.

The system 5000 comprises one or more sensors 5002, circuitry 5004 and one or more feedback devices 5006.

The one or more sensors 5002 of system 5000 are configured to sense a weight distribution of a user 5008. In this example, the one or more sensors 5002 are included within a device 5010. Therefore, the one or more sensors are configured to sense the weight distribution of the user who is positioned on the device 5010. However, in other examples, the one or more sensors 5002 may be provided externally to the device 5010 (described in more detail below).

The circuitry 5004 of system 5000 is configured to analyze a change of weight distribution of the user to determine a posture of the user (e.g. how the user is sitting or how the user is standing).

Finally, the one or more feedback devices 5006 of system 5000 are configured to provide feedback related to the posture of the user when the change in weight distribution of the user satisfies a predetermined condition.

In this manner, the weight distribution of a user can be monitored (e.g. as they are using a device such as an entertainment system). Accordingly, it is possible to verify that a user is correctly positioned and has a correct posture when using the device. As such, it becomes easier for a user to correct their posture (e.g. based on the feedback which is provided). Thus, comfort of the user when using a device can be improved.

In some examples, the system 5000 may be provided as part of the entertainment system 10 as described with reference to FIG. 2 of the present disclosure. In some examples, one or more other devices may also be provided as part of this system. An example of the one or more other devices which can also be provided as part of this system include a display device (such as a screen or a HMD), a control device (such as controller 80 described with reference to FIG. 3 of the present disclosure) and/or an image capture device. Certain features of the system 5000 (such as the circuitry 5004) may also be implemented as part of the entertainment system 10. In particular, one or more of the A/V port 90, GPU 30, CPU 20, RAM 40, SSD 50, Optical Drive 70 and/or Data port 60 of the entertainment system 10 may be configured to implement one or more features of the system 5000.

In examples, the system 5000 (which may be provided as part of the entertainment system 10) may be configured to monitor the weight distribution of the user as the user is using the entertainment system to consume media content. For example, the system 5000 may monitor the weight distribution (and posture) of the user as the user is playing a videogame on the entertainment system and/or consuming media content (e.g. watching a movie (film) on the entertainment system, browsing the internet on the entertainment system or the like). Then, feedback related to the posture of the user can be provided to the user as the user is using the entertainment system in order to ensures that the user operates the entertainment system correctly (i.e. with the correct posture or position).

Consider an example where a user is using an entertainment system in order to play a videogame. In this example, the user is wearing a HMD whilst playing the videogame on the entertainment system. An image viewed by the user on the HMD may have a different orientation to the ground upon which the user is stood. Therefore, it may be difficult for the user to maintain balance in this situation. However, system 5000 monitors the weight distribution of the user and provides feedback to the user. Therefore, it can be ensured that the user operates the entertainment system correctly (i.e. with the correct posture or position) and thus it is easier for the user to retain their balance. Further details of the system 5000 will now be described.

### Sensors

As explained with reference to FIG. 5, the system for monitoring the weight distribution of the user comprises one or more sensors configured to sense a weight distribution of a user 5008.

In the example of FIG. 5, the device 5010 is a device on which the user is positioned. The device may, in examples, be a device which the user is positioned on when using an information processing apparatus. For example, the device 5010 may be a chair which the user sits on while using an information processing device. Alternatively, the device may be a device the user uses when driving a vehicle. For example, the device 5010 may be a chair which the user sits on while driving a vehicle or travelling in a vehicle.

In some examples, the device 5010 may comprise a mat, a seat or a bed or any component thereof or any fitting therefor. Indeed, in examples, the seat or bed component or fitting may comprise a seat pan, seat pad, seat leg, seat wheel, bed frame, bed frame leg, bed frame wheel or a mattress.

More generally, however, the device 5010 may be any device which the user sits, stands or otherwise positions themselves upon

In some examples, at least the one or more sensors 5002 of the system 5000 may be included within the device 5010. However, the present disclosure is not particularly limited in this regard. In other examples, the sensors 5002 may comprise remote sensing devices configured to sense the weight distribution of the user positioned on the device 5010. For example, the sensors 5002 may include an imaging device which can sense the weight distribution of the user. As such, the sensors may be provided externally to the device 5010. Indeed, in some examples, the one or more sensors 5002 may be provided without the device 5010.

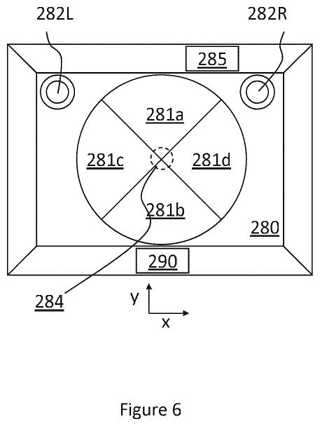

Consider, now, FIG. 6 of the present disclosure. FIG. 6 of the present illustrates an example sensor configuration. The example sensor configuration shown in FIG. 6 is an example where the one or more sensors 5002 of the system 5000 are included in a device 280.

In particular, the device 280 is in the form of a seat cushion 280 (e.g. either integral to a seat, or for placement on a seat), and comprises a central 2D array of 4 sensors 281a-281d arranged at 12 o'clock, 6 o'clock, 9 o'clock and 3 o'clock positions therein respectively, to detect changes in the user's weight in 4 different zones, corresponding to the 4 cardinal directions, and thereby sense a change in weight distribution of the user on the device 280, e.g. as they lean in any direction, generating a directional weight bias. The sensors may be of any suitable type, such as a force sensor, a capacitance pressure sensor, resistance pressure sensor, piezoresistive pressure sensor, piezoelectric pressure sensor, optical pressure sensor, and/or an elastoresistive pressure sensor.

The device 280 may further comprise two input buttons 282L, R to provide action inputs to a computing system (such as entertainment system 200), corresponding to any action button such as any of the action buttons 82R of the handheld controller 80. Furthermore, the device 280 may additionally comprises a haptic feedback device 290, configured to provide haptic feedback to the user as they interact with the computing system 210 via the device 280. The system may optionally comprise any number of additional input devices, actuators or haptic feedback devices, such as buttons and/or an additional joystick, steering or pointer (mouse) control device which may be used in conjunction with the weight distribution sensing system, particularly for users who typically can only control one directional input at a time. For example, the system may optionally comprise gross body motion buttons or controls, such as force/pressure pads on sides of the seat for actuation by the user's thighs, or on the back of the cushion or chair for actuation by shoulders.

In FIG. 6, the two-dimensional (2D), (x, y) array of sensors 281a-d are configured to sense a change in weight distribution in at least two (x, y) dimensions. As the user moves, the sensors 281a-d detect a relative change (increase or decrease) in weight (force/pressure) and thus an overall change in weight distribution can be determined. For example, for a single axis change in weight distribution, if the force/pressure sensed by the first sensor 281a at 12 o'clock increases and the force/pressure sensed by the second sensor 281b on the same (y) axis at 6 o'clock decreases, then the user has shifted their weight forward on that (y) axis, from the back of the cushion to the front of the cushion (e.g. by leaning forward). Similarly, if the force/pressure sensed by the third sensor 281c at 9 o'clock increases and the force/pressure sensed by the fourth sensor 281d at 3 o'clock on the same (x) axis decreases, then the user has shifted their weight backwards on the (x) axis, from the right of the cushion to the left of the cushion (e.g. by leaning left). Of course, multi-axis movements can similarly be detected as combinations of increasing and decreasing sensed force/pressure in multiple axes.

In some embodiments, the device 280 may further comprises a rotational sensor (not shown) for detecting rotation of the user and/or the device 280. Such a rotational sensor can provide additional data about the user's movement e.g. about a third, z-axis. An example of such a sensor may be a potentiometer in a support column of a chair, or a gyroscope.

Parameters of the detection of changes in weight distribution can be customized to suit the user and advantageously, the device 280. In particular, the number and/or arrangement of the sensors 281 can be determined to suit requirements: for example, a mattress device 280 might require a 2D array of sensors that is rectangular, to accommodate a user lying thereon. Furthermore, increasing the number of sensors 281 can increase the granularity of sensed movement changes.

It should be appreciated that the system may not necessarily need pre-calibration and/or may auto-calibrate. For example, the FIG. 6 arrangement with a 2D array of sensors 281a-d is configured to sense a weight distribution in at least two (x, y) dimensions. When the user first positions themselves (e.g. sits) on the device 280, this can automatically be set as a neutral reference position for each sensor 281. If, for example, force sensors are used, then system can measure the total force experienced and equate this total force to the user's weight, and then apply predetermined thresholds for changes in weight distribution e.g. Accordingly, as the user moves, the force sensors 281a-d detect a relative change (increase or decrease) in force and thus an overall change or shift in weight distribution can be determined.

In examples, the one or more sensors: (a) are configured to sense a weight distribution in at least two (x,y) dimensions; and/or (b) comprise a two-dimensional (x, y) array of sensors; and/or (c) are located on, embedded in or located underneath a surface of the device on which the user is positioned in use; and/or (d) comprise one or more sensors which are movable with respect to the input device; (e) comprise one or more force sensors; (f) comprise one or more pressure sensors; and/or (g) comprise one or more: capacitance pressure sensors; resistance pressure sensors; piezoresistive pressure sensors; piezoelectric pressure sensors; optical pressure sensors; and/or elastoresistive pressure sensors.

However, the present disclosure is not particularly limited in this regard. More generally, the one or more sensors may comprise any sensor configured to sense a weight distribution of a user.

### Circuitry

As explained with reference to FIG. 5 of the present disclosure, system 5000 further comprises circuitry 5004. The circuitry 5004 is configured to analyse a change of weight distribution of the user to determine a posture of the user.

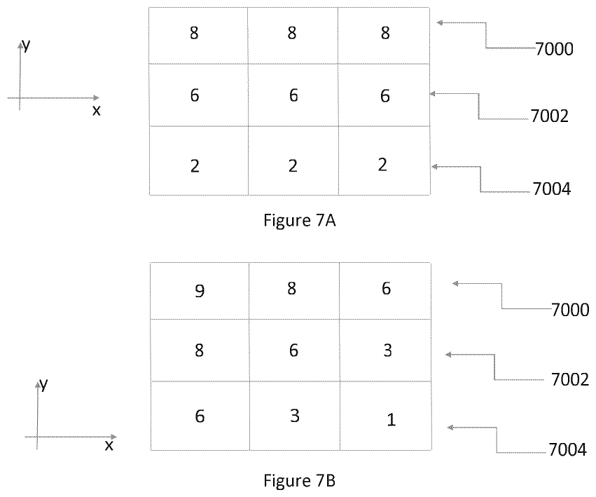

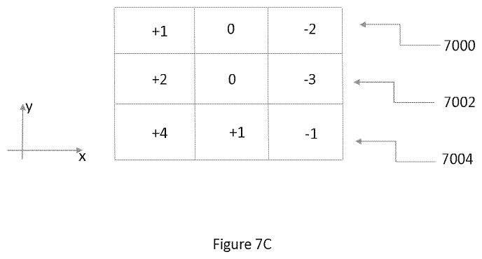

Consider now the example of FIG. 7A of the present disclosure. FIG. 7A of the present disclosure illustrates an example weight distribution in accordance with embodiments of the disclosure.

In this example, an array of sensing devices is shown. The array of sensing devices is an example of one or more sensors configured to sense a weight distribution of a user (for example, a user positioned on a device such as a chair or the like).

The example array of sensing devices shown in FIG. 7A comprises a 3×3 grid of sensing devices. That is, there are 3 rows and 3 columns of sensing devices in this example. However, the present disclosure is not particularly limited in this regard. More generally, the system may include one or more sensors.

The example array of sensing devices may be included within the device on which the user is positioned in use. For example, the example array of sensing devices may be included in device 5010 as described with reference to FIG. 5 of the present disclosure.

Each of the sensing devices shown in FIG. 7A is configured to provide a sensing result. In this example, the sensing result is the weight of the user (e.g. from the total force experienced by the user). Therefore, the array of sensing devices shown in FIG. 7A together provide a measure of the distribution of the weight of the user over the device 5010.

In this example, the individual sensing devices each provide a value indicating the weight of the user experienced by that sensing device. In this example (and merely for illustration purposes) the value is a relative value between 0 to 10 which indicates the relative amount of the weight of the user experienced by that sensing device. In other examples, the sensing devices may provide an absolute value (such as a value for the total force experienced by the sensing device or the like). However, in this example, a low value indicates that a relatively low amount of force (and thus a relatively low percentage of the user's weight) has been sensed by the sensing device. A high value indicates that a relatively high amount of force (and thus a relatively high percentage of the user's weight) has been sensed by the sensing device.

The control device is thus configured to compare the values from the sensing devices in order to identify the weight distribution of the user.

In the example of FIG. 7A, the user's weight is evenly distributed across the x-axis. That is, for a given row, there is no variation in the value detected by the sensing devices for different columns across that row. However, in the example of FIG. 7A, there is a variation in the distribution of the user's weight in the y-axis. That is, the values of the first row 7000 are higher than the values of the second row 7002. However, the values of the second row 7002 are higher than the values of the third row 7004.

FIG. 7A therefore illustrates an example where the user's weight is evenly distributed across the x-axis, and where the user's weight is distributed such that more weight detected by the first row of sensing devices 7000 than either of the second or third rows of sensing devices 7002 and 7004.

A weight distribution such as that illustrated in FIG. 7A of the present disclosure may be observed, for example, when a user is seated, leaning slightly forward, in a chair. In this example, more of the user's weight is distributed over the front of the chair (corresponding to the first row of sensing devices 7000) than the rear of the chair (corresponding to the third row of sensing devices 7006).

The circuitry 5004 may be configured to obtain sensing data from the one or more sensors of the system 5000 at a number of periods of time. For example, the circuitry may periodically obtain sensing data from the one or more sensors of the system 5000. Alternatively, the circuitry 5004 may obtain sensing data from the one or more sensors at predetermined intervals. Alternatively, the circuitry 5004 may obtain sensing data from the one or more sensors of the system 5000 continuously. Alternatively, the circuitry 5004 may obtain sensing data from the one or more sensors of the system at every read-out of the sensors.

Accordingly, the example of FIG. 7A may correspond to data received from the sensing devices at a first instance of time (e.g. when a user has been sat in the chair for only a short period of time).

By comparing the sensing data as received from the one or more sensors of the system 5000 at different instances of time, the circuitry may analyze the change of weight distribution of the user.

Consider, now, FIG. 7B of the present disclosure. FIG. 7B of the present disclosure illustrates an example weight distribution in accordance with embodiments of the disclosure.

In this example, an array of sensing devices is shown. The array of sensing devices is an example of one or more sensors configured to sense a weight distribution of a user.

The example array of sensing devices shown in FIG. 7B comprises a 3×3 grid of sensing devices. That is, there are 3 rows and 3 columns of sensing devices in this example. However, the present disclosure is not particularly limited in this regard. More generally, the system may include one or more sensors.

The example array of sensing devices shown in FIG. 7B is the same as the example array of sensing devices shown in FIG. 7A of the present disclosure. However, FIG. 7B shows the sensing data sensed by the sensing devices at a second time instance of time different to the first instance (corresponding to the sensing data of FIG. 7A). That is, FIG. 7B shows the sensing data sensed by the sensing devices at a different time than shown in FIG. 7A. The second instance of time may be a time after the first instance of time.

The values sensed by the sensing devices illustrated in FIG. 7B (at the second instance of time) are different from the values sensed by the sensing devices as illustrated in FIG. 7A (at the first instance of time). This may be because the user has changed their posture or position on the device 5010. That is, at the user changes their posture or position on the device 5010, this change will be reflected in a change of weight distribution sensed by the sensing devices.

In particular, the values of the weight distribution sensed by the sensing devices as illustrated in FIG. 7B show that the user's weight is no longer evenly distributed across the x-axis. Instead, the user's weight is concentrated in the first row and first column of the sensing array.

The example weight distribution illustrated in FIG. 7B is a weight distribution as may be observed when a user is seated on a chair, leaning slightly forward and to one side of the chair.

As illustrated in FIGS. 7A and 7B of the disclosure, different weight distributions may be observed at different instances of time (e.g. as the user changes their posture or position on the device 5010).

The circuitry 5004 of the system 5000 is configured to analyze a change in the weight distribution of the user. The change in the weight distribution can, in some examples, be analyzed by comparing the weight distribution of the user at two or more instances of time.

Consider, now, the example of FIG. 7C of the present disclosure. The example of FIG. 7C illustrates an example change in weight distribution. This change in weight distribution may be obtained by the circuitry 5004 by analyzing the weight distribution obtained at the first instance of time (as illustrated in FIG. 7A) and the second instance of time (as illustrated in FIG. 7B).

More specifically, the example of FIG. 7C shows the same example array of sensing devices as has been described with reference to FIGS. 7A and 7B. However, the values illustrated in FIG. 7C show the delta or change in sensing values between the first instance of time (as illustrated in FIG. 7A) and the second instance of time (as illustrated in FIG. 7B).

Computing the delta or change in sensing values between the first instance of time and the second instance of time enables the circuitry 5004 to analyze how the weight distribution of the user has changed.

While the example of FIG. 7C uses a simple sum to show the difference for each sensing device, it will be appreciated that the present disclosure is not particularly limited in this regard. For example, the circuitry 5004 may compute an average change in the weight distribution of the user between the two instances of time. Alternatively, the circuitry may compute the similarity between the weight distribution of the user between the two instances of time. Alternatively, the circuitry may compute the absolute sum of the delta values obtained by comparison of the weight distribution of the user at the first and second instance of time. Alternatively, the circuitry 5004 may compute the rate of change of the weight distribution of the user. More generally, the circuitry may perform any suitable analysis of the sensing data obtained from the sensing devices in order to analyse the change of weight distribution of the user. In some examples, the first instance of time (such as that illustrated in FIG. 7A) may be an instance of time corresponding to when the user first positioned themselves on the device (i.e. an initial weight distribution). Comparison with the first instance of time in this case enables the circuitry 5004 to analyze how the weight distribution of the user has changed since this initial weight distribution.

In other examples, the first instance of time (such as that illustrated in FIG. 7A) may be a weight distribution recorded by the user during a calibration phase. That is, during a calibration phase, the circuitry may record the weight distribution of the user on the device. Then, through comparison of a current weight distribution with that of the calibration phase, the circuity 5004 can analyze how the weight distribution of the user has changed since calibration. By using different calibration phases for different users, the circuitry 5004 is able to analyze how the weight distribution of the user has changed in comparison to the way in which the weight distribution of the user was detected during calibration. Thus, the analysis can be tailored to specific users of the device.

In some examples, the calibration phase may correspond to a training phase for a user. That is, in some examples, a user may perform a calibration phase where they are instructed to position themselves on the device (e.g. a gaming chair) with a posture that is demonstrated to them. The data from the one or more sensors may then provide a baseline weight distribution of the user when they are sitting with this instructed posture. Comparison of the weight distribution of the user at a current time with this baseline weight distribution from the first instance of time, enables variation of the weight distribution of the user from the instructed position to be identified (and thus, the posture of the user can be determined).

Hence, in examples, the system may be configured to instruct the user to use the device with a predetermined posture (e.g. by providing a feedback (described in more detail below)) and calibrate the one or more sensors in accordance with the weight distribution of the user when the user occupies the predetermined posture.

Furthermore, the circuitry 5004 of system 5000 is configured to determine a posture of the user in dependence upon the change of weight distribution of the user. For example, the weight distribution of the user at a first instance of time may correspond to a weight distribution of the user when seated or standing with a first posture (such as a weight distribution recorded during the training or calibration phase, where the user was sitting or standing with a first posture). Then, by analysing how the weight distribution changes, the circuitry 5004 can determine how the posture of user has changed from this first posture. An increase in the weight distribution towards row 7000 of the example of FIG. 7A and 7B may show that user is leaning forward, for example (compared to their posture in the training or calibration phase).

In some examples, the system 5000 may use a model of human anatomy in order to determine the posture of the user (how the user is sitting) from the weight distribution or change in weight distribution which has been detected. In some examples, the system may use a trained model (trained on historical or simulated data) to determine the posture of the user in dependence on the change of weight distribution which has been detected. In some examples, the system 5000 may be further configured to determine the posture of the user in accordance with at least one of data (such as sensor data) from a control device held by a user (e.g. the handheld controller 80 described with reference to FIG. 3), data from a wearable device worn by the user in use (e.g. a smart watch or the like), data from a HMD, and/or image data from an image capture device (e.g. an image capture device included in, or as part of, the entertainment system 10 or HMD 802 described with reference to FIGS. 2 and 3 of the present disclosure). The data from the auxiliary device can be used in addition to the detected change in weight distribution of the user.

For example, accelerometers in an auxiliary device (such as a handheld controller) can provide additional information on how the user holds auxiliary device (e.g. whether or not the controller is held parallel to the floor). Alternatively, an image capture device (such as a camera included as part of an entertainment system) can be used to provide additional information on how the user is sitting (e.g. whether or not the user is looking towards a display device (that is, whether or not they are looking at the display device “square-on”, or whether they are “twisted”)).

Accordingly, data from an auxiliary device can be used by the system 5000 to further relate the mass distribution of the user to a body pose of the user (e.g. as evidenced by the position of the controller held by the user). Thus, use of auxiliary data in the determination of the posture of the user can further improve the accuracy by which the posture of the user can be identified.

Furthermore, in some examples, the system 5000 may be configured to detect a change of user positioned on the device, and calibrate the one or more sensors in accordance with the change of user. That is, different users may have different ways of positioning themselves on the device. Therefore, it may be important to obtain a different initial or baseline weight distribution for different users. Accordingly, when a change of user is detected, it may be advantageous to recalibrate the device. This can further improve accuracy and reliability. The way in which system 5000 detects a change of user is not particularly limited. However, in some examples, the system may detect a change of user based on an image or image data received from an image capture device. In other examples, the system 5000 may detect a change of user in accordance with a user operation. In other examples, the system 5000 may detect a change of user in accordance with a signal from an auxiliary device (e.g. a signal from a mobile telephone device or a signal from a wearable device linked with a specific user). In other examples, the system may detect a change of user in accordance with at least one of a total weight or a weight distribution of the user. That is, in some examples, the weight and/or weight distribution of a user can be linked to a user profile and can thus be used to identify a change of user using the device.

Hence, in examples, the system 5000 may be configured to detect a change of user positioned on the device in accordance with at least one of: input from an image capture device, input from an audio capture device, input from the one or more sensors, input from an auxiliary device input from an entertainment device, a detected total weight and/or a detected weight distribution.

Furthermore, in other examples, the circuitry 5004 may be configured to analyze a change of weight distribution of the user by comparison of a current weight distribution of the user with a target weight distribution of the user. The target weight distribution of the user may correspond to an ideal or recommended weight distribution of the user—such as a weight distribution as would be obtained if the user positioned themselves on the device with a correct posture or position. Thus, the analysis identify by how much the weight distribution of the user varies from a target weight distribution.

In other examples, the circuitry may be configured to analyze a change in weight distribution of the user on a rolling basis (i.e. as new sensing data is obtained). That is, in some examples, the circuitry 5004 may be configured to compare new sensing data obtained from the one or more sensors with the most recent data previously obtained from the one or more sensors. This enables the circuitry 5004 to analyze how the weight distribution of the user changes over time. For example, if the user remains in the same posture or position, the comparison of the change in sensing data will show little variation in the weight distribution of the user (e.g. the delta values described with respect to FIG. 7C of the present disclosure would tend to zero). Alternatively, for example, if the user starts to lean to one side, the circuitry 5004 will be able to identify this from analysis of the change in weight distribution. Accordingly, the system 5000 will be able to provide feedback (described in more detail below) as an intervention before the weight distribution of the user deviates too significantly from a target weight distribution. Thus, early intervention can be provided, which can further improve the comfort of the user (by ensuring that the user continues using the device correctly).

Hence, more generally, the circuitry 5004 is configured to analyze a change in weight distribution of the user to determine a posture of the user.

### Feedback Devices

As described with reference to FIG. 5 of the present disclosure, the system 5000 further comprises one or more feedback devices 5006 of system 5000 are configured to provide feedback related to the posture of the user when the change in weight distribution of the user satisfies a predetermined condition. By providing feedback related to the posture of the user in this manner, it is easier for the user to retain or correct their posture. This ensures that the user is (and remains) correctly positioned with the correct posture when using a device (e.g. an entertainment system) and thus improves the comfort of the user.

Identification as to whether the change in weight distribution of the user satisfies a predetermined condition can be performed by the one or more feedback devices 5006. Alternatively, analysis as to whether the change in weight distribution of the user satisfies a predetermined condition can be performed by the circuitry 5004 of the system 5000. Alternatively, analysis as to whether the change in weight distribution of the user satisfies a predetermined condition can be performed by an external server or an external device. For example, the system 5000 may provide a result of analysis of the change in weight distribution of the user to an external device (such as, in some examples, an external entertainment system 10). Then, the system 5000 may receive information from the external device as to whether the change in weight distribution of the user satisfies the predetermined condition. The present disclosure is not particularly limited in this respect.

The predetermined condition is a condition which can be used in order to cause or trigger a feedback to be provided to the user. Therefore, the predetermined condition is a condition set in advance which, if the condition is met, causes or otherwise triggers the feedback to be provided to the user. As such, the specific predetermined condition which is used is not particularly limited and will vary depending upon the situation to which the embodiments of the disclosure are applied.

In some examples, the predetermined condition may be a condition that the change in weight distribution of the user exceeds a threshold value. Consider an example situation where a user is using an entertainment system 10 while sitting on a device such as a gaming chair. In this example, it will be appreciated that small fluctuations in the weight distribution of the user may occur over time (e.g. when compared to an initial or ideal seating position). Accordingly, it may not be necessary to provide a user with feedback if the change in weight distribution of the user is small. This suppresses an increase in user frustration which may be caused by providing unnecessary feedback to the user (e.g. if the change in weight distribution is very small). However, if the weight distribution of the user changes significantly from the initial or ideal seating position, there may be a risk that the user will become uncomfortable (as it can be determined that their posture has changed significantly from a correct posture). As such, if the change in weight distribution of the user changes significantly from the initial or ideal seating position, it may be advantageous to provide the user with feedback in order that the user can correct their position or posture on the gaming chair. Thus, a predetermined threshold value can be used in order to control provision of feedback to the user depending on the magnitude of the change in weight distribution of the user.

In the case of a plurality of sensors being provided in the system 5000, comparison of the change of weight distribution with the threshold value can be made on a sensor by sensor basis. Alternatively, comparison can be made on a combined basis (e.g. using the absolute sum or average value of a plurality of the sensors of the sensing device).

In some examples, the predetermined condition may be a condition that a change in the weight distribution of the user has not exceeded a threshold value for a predetermined period of time.

Consider, again, an example where a user is using an entertainment system 10 while sitting on a device such as a gaming chair. It is known that sitting in the same position (i.e. with the same posture) for too long may lead to the user feeling uncomfortable. Therefore, may be desirable that a user periodically changes their posture or position on the gaming chair. As such, in some examples, if the user has remained in the same position (i.e. if the change in weight distribution of the user is below a threshold amount) for a certain predetermined period of time (e.g. 5 minutes) then it may be advantageous for feedback to be provided to the user in order to cause the user to change their posture or position. Of course, while 5 minutes is provided as an example period of time, it will be appreciated that the predetermined period of time may be significantly shorter or significantly longer than this specific example.

In some examples, the predetermined condition may be a condition that the weight distribution of the user, as changed, corresponds to a predetermined weight distribution. Taking the example of a user sitting on a chair while using an entertainment system 10, it may be known that when a user becomes tired, they begin to slouch on the chair. If the user remains in a slouched position for an extended period of time, they may begin to experience discomfort. Accordingly, when a change in weight distribution is detected (e.g. a change from an initial or ideal position) the weight distribution may be compared to a weight distribution corresponding to a user in a slouched position (or other predetermined weight distribution). This comparison can be performed by pattern analysis, for example. Then, if a weight distribution corresponding to the slouched position (or other predetermined weight distribution) is detected, the feedback may be provided to the user.

The predetermined weight distribution is not particularly limited to a weight distribution corresponding to a user sitting in a slouched position. More generally, any predetermined weight distribution can be used to trigger provision of feedback to a user as required. Indeed, more generally, in some examples the predetermined condition used to trigger provision of the feedback to the user may be a condition that the weight distribution of the user corresponds to a predetermined posture. The predetermined posture may, for example, be a posture in which, if the user remained for an extended period of time, the user would experience discomfort.

Thus, the predetermined condition is any condition which can be used to trigger provision of feedback to the user in accordance with the change of weight distribution which has been detected.

Returning now to FIG. 5 of the present disclosure, the one or more feedback devices 5006 may include any device which can be used in order to provide feedback related to the posture of the user. The feedback devices can provide this feedback to the user. That is, in examples, the feedback may be provided to the user in order to encourage or otherwise cause the user to change their weight distribution (and thus correct their posture). Therefore, any suitable feedback device can be used in order to provide feedback to a user in order to encourage or otherwise cause the user to change their weight distribution.

In examples, the one or more feedback devices are configured to provide at least one of a visual, textual, aural and/or haptic feedback to the user. An example of a visual feedback device includes a light which is illuminated to provide feedback to the user (e.g. a warning light which is illuminated when the weight distribution of the user satisfies a predetermined condition). Alternatively, a further example of a visual feedback device includes a screen or other display device which is configured to display feedback which can be seen by the user.

In some examples, the feedback related to the posture of the user may include an instruction instructing the user how to change their weight distribution to correct their posture. For example, the user may be told to “sit up”, or “lean forward”. Furthermore, in some examples, the instruction can include visual information (such as a picture, video or guide) displayed to the user, demonstrating how the user should position themselves in order to change their weight distribution (e.g. a picture of a person using the device with the correct posture and position). This makes it easier for the user to correct their weight distribution on the device (such that a correct posture or position is attained).

In addition, a screen or other display device can also be used in order to display a message to the user as feedback. In examples, this message can include textual feedback (such as text which can be read by the user on the display device). Thus, in examples, the one or more feedback devices can be configured to provide textual feedback to the user. For example, the message may include a warning such as “you are sitting with bad posture” or “you are leaning too far forwards”. The warning may also include a warning that the current posture may lead to certain levels of discomfort being experienced. This can further encourage the user to change their posture and reduce any associated risk of injury.

An example of aural feedback includes a sound generated by an audio generating device (such as a speaker or the like). The aural feedback can include, for example, a sound, a tone or a message which can be heard by the user. In some examples, the aural feedback can vary depending on the predetermined condition which has been satisfied (e.g. a first sound can be generated if a first predetermined condition is met, while a second, different, sound can be generated if a second, different, predetermined condition is met). Thus, the user can easily understand from the aural feedback how to change or correct their posture.

An example of a haptic feedback device is any device which can be configured to provide haptic feedback to the user. Haptic feedback devices provide tactile information by applying forces, vibrations or touches to the user. The haptic device forming part of system 5000 may be included in the device 5010. However, in other examples, the haptic device may be provided in any other suitable component of the system. For example, a haptic device in a controller such as the handheld controller 80 described with reference to FIG. 3 of the present disclosure can be used in order to provide haptic feedback to the user in accordance with embodiments of the disclosure.

The type and nature of the feedback which is provided may vary depending on the predetermined condition which has been satisfied by the change in weight distribution of the user. In general, however, the feedback is provided in order to prompt the user to change their position on the device (e.g. in order to encourage the user to correct their posture). Hence for example the prompt to the user may be to restore their weight distribution to a predetermined correct weight distribution, the weight distribution being the sensed proxy for their posture.

Consider, again, an example situation of a user using an entertainment system 10 while sitting on a chair such as a gaming chair or the like. In this example, the gaming chair may be provided with a plurality of sensors disposed at different positions thereon. Then, the weight distribution of the user may be recorded when the user is sitting in a target position on the chair (e.g. during an initial training phase). Then, during subsequent use, if the user sits with a different posture in the chair, the system 5000 may detect that the change in the weight distribution of the user satisfies a predetermined condition (e.g. corresponds to a position of bad posture, or varies significantly from the target position). As such, feedback may be provided to prompt the user to correct their posture. The feedback may include, for example, an instruction instructing the user to correct their posture. Alternatively, the feedback may include a warning or other indication to inform the user that a change in posture is required.

In the case of haptic feedback, a plurality of haptic actuators may be disposed at different positions of the chair, and certain haptic actuators may be triggered to prompt the user to not sit with that posture. For example, a user leaning too much on the left side of the chair may feel a haptic response in the left side of the chair. This may prompt the user to correct their posture on the chair.

Thus, comfort of the user when using a device can be improved.

In examples, the level or intensity of the feedback can be increased depending on the type of predetermined condition which has been met. Alternatively, in some examples, the intensity of the feedback is increased when a change in weight distribution of the user is below a threshold value a predetermined time after the feedback has been provided to the user. That is, if feedback is provided to the user in order to encourage the user to change their position, and yet no change in the position of the weight distribution of the user is observed (i.e. if the user has not changed their position as a result of the feedback), then the intensity of the feedback can be increased. This makes it more likely that a user will improve their posture or position on the device even if they do not initially change their position as a result of the feedback which has been provided.

In examples, one or more actuators may be provided as part of the system 5000. The one or more actuators may be configured to change a position and/or orientation of the device on which the user is positioned in use (such as device 5010). The actuators may be configured to change a position and/or orientation of the device in accordance with the feedback provided to the user. That is, consider the example where the feedback is generated in order to warn the user that their weight distribution indicates they are using the device 5010 incorrectly (e.g. leaning too far back). In this example, the one or more actuators may be configured to change a position or orientation of the device in order to correct the position of the user on the device. In the specific example whereby a user is leaning too far back, the one or more actuators may cause the device 5010 to tilt forwards slightly in order that the weight distribution of the user on the device is corrected. This enables the system 5000 to automatically correct a weight distribution of the user in response to the feedback which has been provided.

### Method

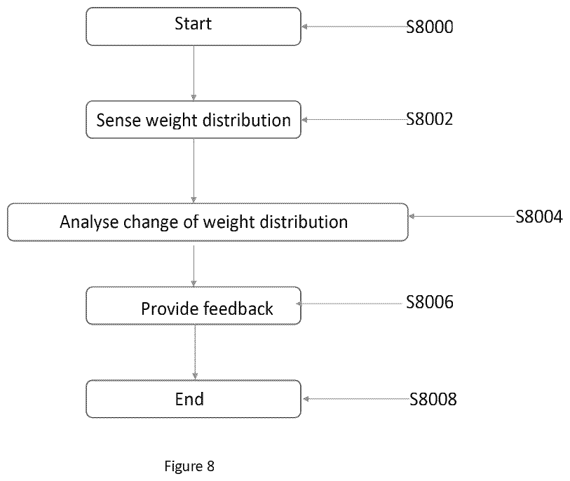

Hence, more generally, a method of monitoring the weight distribution of a user is provided. FIG. 8 illustrates an example method in accordance with embodiments of the disclosure. The example method of FIG. 8 may, optionally, be carried out by a device such as apparatus 1000 of the present disclosure. Alternatively, the example method of FIG. 8 may, optionally, be carried out by a device such as entertainment system 10 of the present disclosure.

The method of FIG. 8 starts at step S8000 and proceeds to step S8002. In step S8002, the method comprises sensing, based on the output from one or more sensors, a change in weight distribution of a user. The method then proceeds to step S8004. In step S8004, the method comprises analysing a change of weight distribution of the user to determine a posture of the user. Then, the method proceeds to step S8006. In step S8006, the method comprises providing feedback, using one or more feedback devices related to the posture of the user when the change in weight distribution of the user satisfies a predetermined condition. The method then proceeds to, and ends with, step S8008.

Of course, it will be appreciated that the embodiments of the present disclosure are not particularly limited to the specific example method illustrated in FIG. 8 of the present disclosure. For example, while the steps of the method of FIG. 8 are illustrated in a particular order, the present disclosure is not limited in this regard. One or more additional method steps may also be included in this method. The method steps shown in FIG. 8 and the one or more additional method steps may be performed in parallel.

For example, the analysis of the change of weight distribution may be performed on a continual basis as new sensor data of the weight distribution of the user is obtained. Furthermore, if the change of weight distribution does not meet the predetermined condition, the method may return to step S8002 and continue in a loop until such a time that a change in weight distribution satisfies the predetermined condition. Furthermore, even if a change meeting a predetermined condition is identified (such that feedback relating to the posture of the user is provided to the user) the method may include continuing to sense and analyze the weight distribution of the user (e.g. in order to identify whether or not the predetermined condition continues to be met).

Thus, in accordance with embodiments of the disclosure, the weight distribution of a user using a device can be monitored. Accordingly, it is possible to verify that a user is correctly positioned when using a device. Moreover, by providing feedback related to the posture of the user, it becomes easier for a user to correct their posture when using the device. As such, comfort of the user when using an information processing apparatus, using a device and/or performing other tasks and operations can be improved.

While a number of embodiments of the disclosure have been described with reference a gaming chair and an entertainment system, it will be appreciated that the present disclosure is not particularly limited in this regard. Alternatively, for example, embodiments of the disclosure can also be applied to a mat, a seat or bed. Alternatively, embodiments of the disclosure may be applied to a surface such as a floor or floor covering (such as a tile, carpet tile or the like). Embodiments of the disclosure can also be applied to a vehicle or other device (such as fitness equipment or the like).

Furthermore, it will be appreciated that the above methods may be carried out on conventional hardware (such as that described previously herein) suitably adapted as applicable by software instruction or by the inclusion or substitution of dedicated hardware. Thus, the required adaptation to existing parts of a conventional equivalent device may be implemented in the form of a computer program product comprising processor implementable instructions stored on a non-transitory machine-readable medium such as a floppy disk, optical disk, hard disk, PROM, RAM, flash memory or any combination of these or other storage media, or realized in hardware as an ASIC (application specific integrated circuit) or an FPGA (field programmable gate array) or other configurable circuit suitable to use in adapting the conventional equivalent device. Separately, such a computer program may be transmitted via data signals on a network such as an Ethernet, a wireless network, the Internet, or any combination of these or other networks.

In addition, embodiments of the present disclosure may be arranged in accordance with the following numbered clauses:

Clause 1) A system for monitoring a weight distribution of a user, the system comprising: one or more sensors configured to sense a weight distribution of a user; circuitry configured to analyse a change of weight distribution of the user to determine a posture of the user; and one or more feedback devices configured to provide feedback related to the posture of the user when the change in weight distribution of the user satisfies a predetermined condition.

Clause 2) The system according to clause 1, wherein the one or more feedback devices are configured to provide at least one of a visual, textual, aural and/or haptic feedback to the user as feedback related to the posture of the user.

Clause 3) The system according to any preceding clause, wherein the intensity of the feedback is increased when a change in weight distribution of the user is below a threshold value a predetermined time after the feedback has been provided to the user.

Clause 4) The system according to any preceding clause, wherein the predetermined condition is a condition that the change in weight distribution of the user exceeds a threshold value.

Clause 5) The system according to any preceding clause, wherein the predetermined condition is a condition that a change in the weight distribution of the user has not exceeded a threshold value for a predetermined period of time.

Clause 6) The system according to any preceding clause, wherein the predetermined condition is a condition that the weight distribution of the user, as changed, corresponds to a predetermined weight distribution.

Clause 7) The system according to any preceding cl clause aim, wherein the feedback related to the posture of the user is feedback to prompt the user to change their weight distribution.

Clause 8) The system according to clause 7, wherein the prompt comprises at least one of an audio, visual and/or textual instruction instructing the user how to change their weight distribution.

Clause 9) The system according to cl clause aim 7, wherein the prompt to the user is to restore their weight distribution to a predetermined correct weight distribution.

Clause 10) The system according to any preceding clause wherein the predetermined condition is a condition that the weight distribution of the user corresponds to a predetermined posture.

Clause 11) The system according to any preceding clause, wherein the circuitry is further configured to determine the posture of the user in accordance with at least one of: data from a control device held by the user, data from a wearable device, data from a head mounted display device, and/or image data from an image capture device.

Clause 12) The system according to any preceding clause, wherein the circuitry is further configured to detect a change of user positioned on the device; and calibrate the one or more sensors in accordance with the change of user.

Clause 13) The system according to clause 12, wherein the circuitry is further configured to detect the change of user positioned on the device in accordance with at least one of: input from an image capture device, input from an audio capture device, input from the one or more sensors, input from an auxiliary device, input from an entertainment device, a detected total weight and/or a detected weight distribution.

Clause 14) The system according to clause 12, wherein to calibrate of the one or more sensors, the circuitry is further configured to: generate a user feedback to instruct the user to use the device with a predetermined posture and calibrate the one or more sensors in accordance with the weight distribution of the user when the user occupies the predetermined posture.

Clause 15) The system according to any preceding clause, wherein the one or more sensors: (a) are configured to sense a weight distribution in at least two (x, y) dimensions; and/or (b) comprise a two-dimensional (x, y) array of sensors; and/or (c) are located on, embedded in or located underneath a surface of the device on which the user is positioned in use; and/or (e) comprise one or more sensors which are movable with respect to the input device; (f) comprise one or more force sensors; (g) comprise one or more pressure sensors; and/or (h) comprise one or more: capacitance pressure sensors; resistance pressure sensors; piezoresistive pressure sensors; piezoelectric pressure sensors; optical pressure sensors; and/or elastoresistive pressure sensors.

Clause 16) The system according to any preceding clause, further comprising one or more additional input devices, actuators or haptic feedback devices.

Clause 17) The system according to any preceding clause, wherein one or more actuators are configured to change a position and/or orientation of the device in response to the feedback provided to the user.

Clause 18) The system according to any preceding clause, wherein the system further comprises an entertainment system.

Clause 19) The systems according to clause 18, wherein the system is configured to monitor the weight distribution of the user as the user operates the entertainment system to play a videogame and/or consume media content.

Clause 20) A mat, a seat or bed or any component thereof or any fitting therefor, comprising the system for monitoring the weight distribution of the user according to any preceding clause.

Clause 21) The seat or bed component or fitting of clause 20, wherein the component or fitting comprises a seat pan, seat pad, seat leg, seat wheel, bed frame, bed frame leg, bed frame wheel or a mattress.

Clause 22) A method of monitoring weight distribution of a user, the method comprising: sensing, based on the output from one or more sensors, a change in weight distribution of a user; analysing a change of weight distribution of the user to determine a posture of the user; and providing feedback related to the posture of the user, using one or more feedback devices, when the change in weight distribution of the user satisfies a predetermined condition.

Clause 23) A computer program comprising instructions which, when implemented by the computer, cause the computer to perform the method of clause 22.

Clause 24) A non-transitory computer readable storage medium storing the computer program of clause 23.

It will be appreciated that numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. A system for monitoring a weight distribution of a user, the system comprising:
one or more sensors configured to sense a weight distribution of a user;
circuitry configured to analyse a change of weight distribution of the user to determine a posture of the user; and
one or more feedback devices configured to provide feedback related to the posture of the user when the change in weight distribution of the user satisfies a predetermined condition.

2. The system according to claim 1, wherein the one or more feedback devices are configured to provide at least one of a visual, textual, aural and/or haptic feedback to the user as feedback related to the posture of the user.

3. The system according to claim 1, wherein the intensity of the feedback is increased when a change in weight distribution of the user is below a threshold value a predetermined time after the feedback has been provided to the user.

4. The system according to claim 1, wherein the predetermined condition is a condition that the change in weight distribution of the user exceeds a threshold value.

5. The system according to claim 1, wherein the predetermined condition is a condition that a change in the weight distribution of the user has not exceeded a threshold value for a predetermined period of time.

6. The system according to claim 1, wherein the predetermined condition is a condition that the weight distribution of the user, as changed, corresponds to a predetermined weight distribution.

7. The system according to claim 1, wherein the feedback related to the posture of the user is feedback to prompt the user to change their weight distribution.

8. The system according to claim 7, wherein the prompt comprises at least one of an audio, visual and/or textual instruction instructing the user how to change their weight distribution.

9. The system according to claim 7, wherein the prompt to the user is to restore their weight distribution to a predetermined correct weight distribution.

10. The system according to claim 1, wherein the predetermined condition is a condition that the weight distribution of the user corresponds to a predetermined posture.

11. The system according to claim 1, wherein the circuitry is further configured to determine the posture of the user in accordance with at least one of: data from a control device held by the user, data from a wearable device, data from a head mounted display device, and/or image data from an image capture device.

12. The system according to claim 1, wherein the circuitry is further configured to detect a change of user positioned on the device; and calibrate the one or more sensors in accordance with the change of user.

13. The system according to claim 12, wherein the circuitry is further configured to detect the change of user positioned on the device in accordance with at least one of: input from an image capture device, input from an audio capture device, input from the one or more sensors, input from an auxiliary device, input from an entertainment device, a detected total weight and/or a detected weight distribution.

14. The system according to claim 12, wherein to calibrate of the one or more sensors, the circuitry is further configured to: generate a user feedback to instruct the user to use the device with a predetermined posture and calibrate the one or more sensors in accordance with the weight distribution of the user when the user occupies the predetermined posture.

15. The system according to claim 1, wherein one or more of:
a. the one or more sensors are configured to sense a weight distribution in at least two (x, y) dimensions;
b. the one or more sensors comprise a two-dimensional (x, y) array of sensors;
c. the one or more sensors are located on, embedded in or located underneath a surface of the device on which the user is positioned in use;
d. the one or more sensors comprise one or more sensors which are movable with respect to the input device;
e. the one or more sensors comprise one or more force sensors;
f. the one or more sensors comprise one or more pressure sensors; and
g. the one or more sensors comprise one or more: capacitance pressure sensors; resistance pressure sensors; piezoresistive pressure sensors; piezoelectric pressure sensors; optical pressure sensors; and/or elastoresistive pressure sensors.

16. The system according to claim 1, further comprising one or more additional input devices, actuators or haptic feedback devices.

17. The system according to claim 1, wherein one or more actuators are configured to change a position and/or orientation of the device in response to the feedback provided to the user.

18. The system according to claim 1, wherein the system further comprises an entertainment system.

19. The system according to claim 18, wherein the system is configured to monitor the weight distribution of the user as the user operates the entertainment system to play a videogame and/or consume media content.

20. An apparatus, comprising:
a mat, a seat, a bed or any component thereof or any fitting therefor, which includes a system for monitoring the weight distribution of a user, where the system includes:
one or more sensors configured to sense a weight distribution of a user;
circuitry configured to analyse a change of weight distribution of the user to determine a posture of the user; and
one or more feedback devices configured to provide feedback related to the posture of the user when the change in weight distribution of the user satisfies a predetermined condition.

21. The apparatus of claim 20, wherein the component or fitting comprises a seat pan, seat pad, seat leg, seat wheel, bed frame, bed frame leg, bed frame wheel or a mattress.

22. A method of monitoring weight distribution of a user, the method comprising:
sensing, based on the output from one or more sensors, a change in weight distribution of a user;
analysing a change of weight distribution of the user to determine a posture of the user; and
providing feedback related to the posture of the user, using one or more feedback devices, when the change in weight distribution of the user satisfies a predetermined condition.

23. A non-transitory machine-readable storage medium containing a computer program comprising instructions which, when implemented by the computer, cause the computer to perform a method of monitoring weight distribution of a user, the method comprising:
sensing, based on the output from one or more sensors, a change in weight distribution of a user;
analysing a change of weight distribution of the user to determine a posture of the user; and
providing feedback related to the posture of the user, using one or more feedback devices, when the change in weight distribution of the user satisfies a predetermined condition.

24. (canceled)
